(19) United States
(12) Patent Application Publication
(10) Pub. No.: US 2024/0228676 A9
(43) Pub. Date: Jul. 11, 2024

(54) GLYCIDYL (METH)ACRYLATE COMPOSITION

(71) Applicant: MITSUBISHI GAS CHEMICAL COMPANY, INC., Tokyo (JP)

(72) Inventors: Michihiro YURI, Niigata (JP); Kouji SUZUKI, Kanagawa (JP); Shu SUZUKI, Kanagawa (JP)

(73) Assignee: MITSUBISHI GAS CHEMICAL COMPANY, INC., Tokyo (JP)

(21) Appl. No.: 18/272,216

(22) PCT Filed: Jan. 19, 2022
(86) PCT No.: PCT/JP2022/001690

§ 371 Date: Jul. 13, 2023

(30) Foreign Application Priority Data
Jan. 20, 2021 (JP) 2021-007129

(51) Int. Cl. C08F 2/40; C08K 5/13

(52) U.S. Cl. CPC C08F 2/40; C08K 5/13

(57) ABSTRACT

Provided are a glycidyl (meth)acrylate composition, which includes a phenolic polymerization inhibitor that is unlikely to deteriorate such that the glycidyl (meth)acrylate composition can be stably stored for a long period of time, and a method for suppressing deactivation of a phenolic polymerization inhibitor in a glycidyl (meth)acrylate resin composition. More specifically, provided are: a glycidyl (meth)acrylate composition including a glycidyl (meth)acrylate, a quaternary ammonium salt, a strong acid salt, and a phenolic polymerization inhibitor; and a method for suppressing deactivation of a phenolic polymerization inhibitor in a glycidyl (meth)acrylate composition, the method including adjusting the content of a strong acid salt in the glycidyl (meth)acrylate composition to a certain amount relative to an amount of a quaternary ammonium salt by mole.

### TECHNICAL FIELD

The present invention relates to a glycidyl (meth)acrylate composition. More particularly, the present invention relates to a glycidyl (meth)acrylate composition, which includes a phenolic polymerization inhibitor that is unlikely to deteriorate such that the glycidyl (meth)acrylate composition can be stably stored for a long period of time. The present invention also provides a method for suppressing deactivation of a phenolic polymerization inhibitor in a glycidyl (meth)acrylate resin composition.

### BACKGROUND ART

Glycidyl (meth)acrylate compositions are widely used as various industrial raw materials such as resin modifiers, thermosetting paints, adhesives, fiber treatment agents, antistatic agents, and ion exchange resins. The term “glycidyl (meth)acrylate” refers to glycidyl acrylate or glycidyl methacrylate in the art.

A representative method for synthesizing glycidyl (meth)acrylate is a method using epichlorohydrin as a raw material. Such methods are roughly classified into the following two methods.

The first one is a method for synthesizing glycidyl (meth)acrylate by reacting epichlorohydrin and an alkali metal salt of (meth)acrylic acid in the presence of a catalyst (Patent Literatures 1 and 2). The second one is a method for synthesizing glycidyl (meth)acrylate by reacting epichlorohydrin and (meth)acrylic acid in the presence of a catalyst, followed by a ring closure reaction with an alkaline aqueous solution (Patent Literature 3). In either method, a quaternary ammonium salt is used as the catalyst.

In addition, 1,3-dichloropropanol is a reaction by-product during the synthesis of glycidyl (meth)acrylate. Since 1,3-dichloropropanol has a boiling point close to that of glycidyl methacrylate and is difficult to separate by distillation, reduction treatment may be performed using a quaternary ammonium salt as a catalyst (Patent Literature 4).

As described above, a quaternary ammonium salt is widely used in the glycidyl (meth)acrylate production process.

Meanwhile, Non Patent Literature 1 teaches that the addition reaction of phenol to epoxy groups proceeds in the presence of a quaternary ammonium salt. Generally, a phenolic polymerization inhibitor such as p-methoxyphenol is used as a polymerization inhibitor for glycidyl (meth)acrylate. Therefore, there is concern that the incorporation of a quaternary ammonium salt into a product during the production process induces a phenolic polymerization inhibitor to react with the epoxy group of glycidyl (meth)acrylate during storage, causing the amount of the phenolic polymerization inhibitor present in a glycidyl (meth)acrylate composition to continuously decreases or causing unintended polymerization to occur.

### CITATION LIST

### Patent Literature

Patent Literature 1: Japanese Patent Publication (Kokai) No. 7-2818 A (1995)
Patent Literature 2: Japanese Patent Publication (Kokai) No. 9-59268 A (1997)
Patent Literature 3: Japanese Patent Publication (Kokai) No. 7-118251 A (1995)
Patent Literature 4: Japanese Patent No. 4666139

### Non Patent Literature

Non Patent Literature 1: Chem. Commun., 2015, 51, 15133-15136

### SUMMARY OF INVENTION

### Technical Problem

Given the above, the present invention provides a glycidyl (meth)acrylate composition, which includes a phenolic polymerization inhibitor that is unlikely to deteriorate (be deactivated) such that the glycidyl (meth)acrylate composition can be stably stored for a long period of time. The present invention also provides a method for suppressing deactivation of a phenolic polymerization inhibitor in a glycidyl (meth)acrylate resin composition.

### Solution to Problem

The present inventors made intensive studies to solve the above-described problems. As a result, the present inventors found that the problems can be solved by adding a strong acid salt to a glycidyl (meth)acrylate composition comprising a quaternary ammonium salt. This has led to the completion of the present invention. Specifically, the present invention is, for example, as follows.
<1> A method for suppressing deactivation of a phenolic polymerization inhibitor in a glycidyl (meth)acrylate composition, comprising adjusting a content of a strong acid salt in the glycidyl (meth)acrylate composition to 0.50 equivalents or more relative to an amount of a quaternary ammonium salt by mole.
<2> The method according to the above <1>, wherein the strong acid salt is selected from the group consisting of a sulfonate, a nitrate, and a phosphate.
<3> The method according to the above <2>, wherein the strong acid salt is alkyl benzene sulfonate or alkyl sulfonate.
<4> The method according to the above <3>, wherein the strong acid salt is sodium p-toluenesulfonate or sodium methanesulfonate.
<5> The method according to the above <2>, wherein the strong acid salt is sodium nitrate.
<6> The method according to any one of the above <1> to <5>, wherein the quaternary ammonium salt is tetraalkylammonium halogenide.
<7> The method according to the above <6>, wherein the quaternary ammonium salt is tetramethylammonium chloride or triethylmethylammonium chloride.
<8> The method according to any one of the above <1> to <7>, wherein the phenolic polymerization inhibitor is p-methoxyphenol, hydroquinone, or Topanol A (2-(tert-butyl)-4,6-dimethylphenol).
<9> The method according to any one of the above <1> to <8>, wherein the glycidyl (meth)acrylate composition comprises the strong acid salt in an amount of 0.50 equivalents or more relative to the amount of the quaternary ammonium salt by mole.
<10> The method according to any one of the above <1> to <9>, wherein the glycidyl (meth)acrylate is glycidyl methacrylate.
<11> A glycidyl (meth)acrylate composition comprising a glycidyl (meth)acrylate, a quaternary ammonium salt, a strong acid salt, and a phenolic polymerization inhibitor.
<12> The glycidyl (meth)acrylate composition according to the above <11>, wherein the strong acid salt is selected from the group consisting of a sulfonate, a nitrate, and a phosphate.
<13> The glycidyl (meth)acrylate composition according to the above <12>, wherein the strong acid salt is alkyl benzene sulfonate or alkyl sulfonate.
<14> The glycidyl (meth)acrylate composition according to the above <13>, wherein the strong acid salt is sodium p-toluenesulfonate or sodium methanesulfonate.
<15> The glycidyl (meth)acrylate composition according to the above <12>, wherein the strong acid salt is sodium nitrate.
<16> The glycidyl (meth)acrylate composition according to any one of the above <11> to <15>, wherein the quaternary ammonium salt is tetraalkylammonium halogenide.
<17> The glycidyl (meth)acrylate composition according to the above <16>, wherein the quaternary ammonium salt is tetramethylammonium chloride or triethylmethylammonium chloride.
<18> The glycidyl (meth)acrylate composition according to any one of the above <11> to <17>, wherein the phenolic polymerization inhibitor is p-methoxyphenol, hydroquinone, or Topanol A (2-(tert-butyl)-4,6-dimethylphenol).
<19> The glycidyl (meth)acrylate composition according to any one of the above <11> to <18>, which comprises the strong acid salt in an amount of 0.50 equivalents or more relative to the amount of the quaternary ammonium salt by mole.
<20> The glycidyl (meth)acrylate composition according to any one of the above <11> to <19>, wherein the glycidyl (meth)acrylate is glycidyl methacrylate.

### Advantageous Effects of Invention

According to the present invention, a glycidyl (meth)acrylate composition, which includes a phenolic polymerization inhibitor that is unlikely to deteriorate (be deactivated) such that the glycidyl (meth)acrylate composition can be stably stored for a long period of time can be provided.

### Solution to Problem

### 1. Glycidyl (Meth)Acrylate Composition

The glycidyl (meth)acrylate composition of the present invention comprises a glycidyl (meth)acrylate, a quaternary ammonium salt, a strong acid salt, and a phenolic polymerization inhibitor. Each component will be described below.

### 1.1 Glycidyl (Meth)Acrylate

The term “glycidyl (meth)acrylate” refers to glycidyl acrylate and glycidyl methacrylate. In one embodiment of the present invention, glycidyl (meth)acrylate may be glycidyl acrylate. In another embodiment of the present invention, glycidyl (meth)acrylate may be glycidyl methacrylate. In a preferred embodiment of the present invention, glycidyl (meth)acrylate is glycidyl methacrylate.

Glycidyl (meth)acrylate can be produced by a known production method. As stated above, examples of a representative glycidyl (meth)acrylate production method include methods using epichlorohydrin (hereinafter also referred to as “EpCH”) as a raw material, which are roughly divided into the following two: a method for synthesizing glycidyl (meth)acrylate by reacting epichlorohydrin and an alkali metal salt of (meth)acrylic acid in the presence of a catalyst (Patent Literatures 1 and 2): and a method for synthesizing glycidyl (meth)acrylate by reacting epichlorohydrin and (meth)acrylic acid in the presence of a catalyst, followed by a ring closure reaction with an alkaline aqueous solution (Patent Literature 3). In these methods, a quaternary ammonium salt is used as a catalyst.

Known substances can be used as quaternary ammonium salts used in these production methods. Examples thereof include: tetraalkylammonium halogenides such as tetramethylammonium chloride (hereinafter also referred to as “TMAC”), trimethylethylammonium chloride, dimethyl diethyl ammonium chloride, triethylmethylammonium chloride (hereinafter also referred to as “EMAC”), and tetraethylammonium chloride; and trialkylbenzylammonium halogenides such as trimethylbenzylammonium chloride and triethylbenzylammonium chloride. One kind or any combination of two or more kinds of the above-described quaternary ammonium salts may be used. Among the above, tetramethylammonium chloride, triethylmethylammonium chloride, tetraethylammonium chloride, triethylbenzylammonium chloride, and trimethylbenzylammonium chloride are preferably used. The amount of the catalyst used is generally 0.01 to 1.5% by mole with respect to (meth)acrylic acid.

In each production method, the synthetic liquid contains a quaternary ammonium salt as a catalyst as well as a large amount of solids such as alkali chloride, which is approximately equimolar to the produced glycidyl (meth)acrylate. In addition, for the purpose of improving the yield, the synthesis reaction is carried out with excess EpCH. Generally, after the completion of the synthesis, it is common to remove the solids from the synthetic liquid by a method such as filtration or washing with water, recover the unreacted surplus EpCH by distillation, and then recover glycidyl (meth)acrylate by distillation. EpCH recovered by distillation is recycled as a synthetic raw material. Hereinafter, the process up to the removal of solids from the synthetic liquid is referred to as the synthesis step, the liquid obtained by removing the solids from the synthetic liquid is referred to as the mother liquor, and the process after the removal of the solids is referred to as the distillation step.

The distillation step may be a batch system or a continuous system, and simple distillation, rectification, thin film distillation, and the like can be appropriately combined. The synthesis stem is carried out preferably in the presence of an appropriate polymerization inhibitor. Known compounds such as phenolic compounds, phenothiazine compounds, N-oxyl compounds, amine compounds, phosphorus compounds, sulfur compounds, and transition metal compounds can be used. It is preferable to use these compounds also in the distillation step. Moreover, polymerization can be further prevented by supplying molecular oxygen as needed. As described above, generally, a phenolic polymerization inhibitor such as p-methoxyphenol is used as a polymerization inhibitor for glycidyl (meth)acrylate.

Since EpCH is used as a raw material in each of the above-described methods, the resulting glycidyl (meth)acrylate contains 1,3-dichloropropanol (hereinafter also referred to as “1,3-DCP”) as an impurity. Since 1,3-DCP has a boiling point very close to that of glycidyl (meth)acrylate, separation by distillation is impractical. In other words, when glycidyl (meth)acrylate is recovered after recovering EpCH in the distillation step as described above, almost all of the 1,3-DCP produced in the synthesis step is recovered with glycidyl (meth)acrylate.

For example, in the purification step of glycidyl methacrylate (hereinafter also referred to as “GMA” in some cases), the addition of a quaternary ammonium salt to crude GMA including 1,3-DCP allows an equilibrium reaction shown in Formula 1 below to proceed, resulting in generation of EpCH and 3-chloro-2-hydroxypropyl methacrylate (hereinafter also referred to as “MACE”). The produced EpCH is a low boiling point component relative to GMA, and MACE has a sufficiently high boiling point relative to GMA.

1,3-DCP+GMA→EpCH+MACE (Formula 1)

Examples of a quaternary ammonium salt to be added in the purification step include: tetraalkylammonium halogenides such as tetramethylammonium chloride, trimethylethylammonium chloride, dimethyl diethyl ammonium chloride, triethylmethylammonium chloride, and tetraethylammonium chloride; and trialkylbenzylammonium halogenides such as trimethylbenzylammonium chloride and triethylbenzylammonium chloride. It is possible to use one kind or two or more kinds of quaternary ammonium salts to be added. Among the above, tetramethylammonium chloride, triethylmethylammonium chloride, tetraethyl ammonium chloride, triethylbenzylammonium chloride, and trimethylbenzylammonium chloride are preferably used. The quaternary ammonium salt to be added may be the same as or different from that used in the synthesis. The amount of the quaternary ammonium salt used is 0.001% to 1%, preferably 0.01% to 0.5%, more preferably 0.02% to 0.4% with respect to crude glycidyl (meth)acrylate. When the amount is less than this, the reaction becomes slow, and when it is more than this, it is economically disadvantageous.

The shape of the quaternary ammonium salt used in the synthesis and purification steps is not particularly limited. The quaternary ammonium salt may be in a powdery or granular solid form or a slurry-dispersed form in an aqueous solution or glycidyl (meth)acrylate in the purification step. The quaternary ammonium salt in a granular or powdery form is usually used.

In addition, a method for adding the quaternary ammonium salt is not particularly limited. In the case of a solid, the quaternary ammonium salt may be charged into a reactor using a hopper or the like, and in the purification step, it may be fed crude glycidyl (meth)acrylate or the like to be added. Although it may be divided and added several times, it is usually added at once.

The purity of glycidyl (meth)acrylate used in the present invention is preferably 97% or more, more preferably 98% or more, still more preferably 99% or more, even more preferably 99.5% or more. The purity of glycidyl (meth)acrylate can be measured by a conventional method such as gas chromatography (GC).

### 1.2 Quaternary Ammonium Salt

As the quaternary ammonium salt, one used as a reaction catalyst in the step of producing glycidyl (meth)acrylate and one added in the purification step may remain in the glycidyl (meth)acrylate composition; thus, it may be present in the glycidyl (meth)acrylate composition.

Examples of a quaternary ammonium salt which may be present in the glycidyl (meth)acrylate composition include: tetraalkylammonium halogenides such as tetramethylammonium chloride, trimethylethylammonium chloride, dimethyl diethyl ammonium chloride, triethylmethylammonium chloride, and tetraethylammonium chloride; and trialkylbenzylammonium halogenides such as trimethylbenzylammonium chloride and triethylbenzylammonium chloride. One kind or any combination of two or more kinds of the above may be a quaternary ammonium salt which may be present in the glycidyl (meth)acrylate composition. Among the above, a quaternary ammonium salt which may be present in the glycidyl (meth)acrylate composition are preferably, tetramethylammonium chloride, triethylmethylammonium chloride, tetraethyl ammonium chloride, triethylbenzylammonium chloride, and trimethylbenzylammonium chloride. In a preferred embodiment, the quaternary ammonium salt which may be present in the glycidyl (meth)acrylate composition is tetraalkylammonium halogenide. In a more preferred embodiment, the quaternary ammonium salt which may be present in the glycidyl (meth)acrylate composition is tetramethylammonium chloride or triethylmethylammonium chloride.

As stated above, the present inventors found that a quaternary ammonium salt which may remain in a glycidyl (meth)acrylate composition or a glycidyl (meth)acrylate product reacts with a phenolic polymerization inhibitor present in a glycidyl (meth)acrylate composition, which causes the phenolic polymerization inhibitor in the reaction system to decrease, impairing the long-term storage stability of the glycidyl (meth)acrylate composition. Therefore, the present invention is intended to ensure the long-term storage stability of a glycidyl (meth)acrylate composition by adjusting the content of a quaternary ammonium salt in the glycidyl (meth)acrylate composition.

The content of the quaternary ammonium salt present in the glycidyl (meth)acrylate composition of the present invention may be 30 ppm or less. In one embodiment of the present invention, the content of the quaternary ammonium salt present in the glycidyl (meth)acrylate composition may be, for example, 30 ppm, 20 ppm, 10 ppm, 9 ppm, 8 ppm, 7 ppm, 6 ppm, 5 ppm, 4 ppm, 3 ppm, 2 ppm, 1 ppm, 0.9 ppm, 0.8 ppm, 0.7 ppm, 0.6 ppm, 0.5 ppm, 0.4 ppm, 0.3 ppm, 0.2 ppm, or 0.1 ppm. The content of the quaternary ammonium salt present in the glycidyl (meth)acrylate composition of the present invention is preferably 10 ppm or less, more preferably 5 ppm or less, still more preferably 4 ppm or less, 3 ppm or less, or 2 ppm or less, even more preferably 1 ppm or less. As long as the content of the quaternary ammonium salt present in the glycidyl (meth)acrylate composition of the present invention is within the above-described range, the reaction between the quaternary ammonium salt and the phenolic polymerization inhibitor can be appropriately suppressed.

### 1.3 Strong Acid Salt

The strong acid salt used in the present invention is not particularly limited as long as it can suppress deactivation of the phenolic polymerization inhibitor present in the glycidyl (meth)acrylate composition. Examples thereof include a sulfonate, a nitrate, and a phosphate.

In one embodiment of the present invention, the strong acid salt is selected from the group consisting of sodium salts, calcium salts, potassium salts, and magnesium salts of the above-described strong acid. In one embodiment of the present invention, the strong acid salt may be a sodium salt of the above-described strong acid. In another embodiment of the present invention, the strong acid salt may be a calcium salt of the above-described strong acid. In a preferred embodiment of the present invention, the strong acid salt is a sodium salt of the above-described strong acid.

In one embodiment of the present invention, the strong acid salt may be a sulfonate. In a preferred embodiment of the present invention, the strong acid salt may be alkyl benzene sulfonate or alkyl sulfonate. In a preferred embodiment of the present invention, the strong acid salt may be, for example, sodium alkylbenzene sulfonate, potassium alkylbenzene sulfonate, calcium bis(alkylbenzenesulfonate), magnesium bis(alkylbenzenesulfonate), sodium alkylsulfonate, potassium alkylsulfonate, calcium bis(alkylsulfonate), or magnesium bis(alkylsulfonate). In a more preferred embodiment of the present invention, the strong acid salt may be, for example, p-toluenesulfonate, methanesulfonate, laurylsulfonate, dodecylbenzenesulfonate, or benzenesulfonate. In a further preferred embodiment of the present invention, the strong acid salt is sodium p-toluenesulfonate (hereinafter also referred to as “p-TSANa”) or sodium methanesulfonate (hereinafter also referred to as “Me-SO₃Na”).

In another embodiment of the present invention, the strong acid salt may be a nitrate. In a preferred embodiment of the present invention, the strong acid salt may be, for example, sodium nitrate (NaNO₃), calcium nitrate, potassium nitrate, or magnesium nitrate. In a more preferred embodiment of the present invention, the strong acid may be sodium nitrate.

In another embodiment of the present invention, the strong acid salt may be a phosphate. In a more preferred embodiment of the present invention, the strong acid salt may be, for example, sodium phosphate, calcium phosphate, potassium phosphate, or magnesium phosphate. In a further preferred embodiment of the present invention, the strong acid salt is sodium phosphate.

In one embodiment of the present invention, the content of the strong acid salt in the glycidyl (meth)acrylate composition is adjusted to 0.50 equivalents or more relative to the amount of the quaternary ammonium salt by mole. The content of the strong acid salt in the glycidyl (meth)acrylate composition may be, for example, 0.50 equivalents, 0.75 equivalents, 1.00 equivalent, 1.25 equivalents, 1.50 equivalents, 1.75 equivalents, 2.00 equivalents, 2.50 equivalents, or 3.00 equivalents relative to the amount of the quaternary ammonium salt by mole. The content of the strong acid salt in the glycidyl (meth)acrylate composition is preferably 0.50 equivalents or more, more preferably 0.75 equivalents or more, still more preferably 1.00 equivalent or more relative to the amount of the quaternary ammonium salt by mole. In one preferred embodiment of the present invention, the content of the strong acid salt in the glycidyl (meth)acrylate composition is adjusted to 0.50 equivalents or more relative to the amount of the quaternary ammonium salt by mole. In one more preferred embodiment of the present invention, the content of the strong acid salt in the glycidyl (meth)acrylate composition is adjusted to 0.75 equivalents or more relative to the amount of the quaternary ammonium salt by mole. In one further preferred embodiment of the present invention, the content of the strong acid salt in the glycidyl (meth)acrylate composition is adjusted to 1.00 equivalent or more relative to the amount of the quaternary ammonium salt by mole. In an embodiment of the present invention, the content of the strong acid salt in the glycidyl (meth)acrylate composition can be appropriately adjusted to, for example, 1.50 equivalents or less, 1.75 equivalents or less, 2.00 equivalents or less, 2.50 equivalents or less, 3.00 equivalents or less, or 5.00 equivalents or less relative to the amount of the quaternary ammonium salt by mole.

### 1.4 Phenolic Polymerization Inhibitor

The phenolic polymerization inhibitor is a polymerization inhibitor that is generally used in producing glycidyl (meth)acrylate, which is present in the produced glycidyl (meth)acrylate composition.

Examples of the phenolic polymerization inhibitor used in producing the glycidyl (meth)acrylate of the present invention include, but are not limited to, p-methoxyphenol (hereinafter also referred to as “MQ”), hydroquinone, 2,6-di-tert-butyl-4-methylphenol, 2,2′-methylene-bis(4-methyl-6-tert-butylphenol), and Topanol A (2-(tert-butyl)-4,6-dimethylphenol). In an embodiment of the present invention, the phenolic polymerization inhibitor is preferably p-methoxyphenol, hydroquinone, or Topanol A (2-(tert-butyl)-4,6-dimethylphenol), more preferably p-methoxyphenol or hydroquinone, most preferably p-methoxyphenol.

The amount of the phenolic polymerization inhibitor used in producing glycidyl (meth)acrylate to be added is generally in a range of 0.0005 to 0.01 equivalents with respect to the amount of (meth)acryloyl group by mole. The content of the phenolic polymerization inhibitor present in the produced glycidyl (meth)acrylate composition is in a range of 20 to 200 ppm, preferably 20 to 150 ppm.

### 2. Method for Suppressing Deactivation of Phenolic Polymerization Inhibitor in Glycidyl (Meth)Acrylate Composition

As stated above, the present inventors found that a quaternary ammonium salt which may remain in a glycidyl (meth)acrylate composition or a glycidyl (meth)acrylate product reacts with a phenolic polymerization inhibitor present in a glycidyl (meth)acrylate composition, which causes the phenolic polymerization inhibitor in the reaction system to decrease. Based on these findings obtained by the present inventors, the present invention also provides a method for suppressing deactivation of a phenolic polymerization inhibitor in a glycidyl (meth)acrylate composition, including adjusting the content of a strong base in the glycidyl (meth)acrylate composition to a certain amount relative to an amount of a quaternary ammonium salt by mole.

In the method for suppressing deactivation of a phenolic polymerization inhibitor in a glycidyl (meth)acrylate composition of the present invention, the content of a quaternary ammonium salt in the glycidyl (meth)acrylate composition may be adjusted to preferably 10 ppm or less, more preferably 5 ppm or less, still more preferably 1 ppm or less. By adjusting the content of the quaternary ammonium salt in the glycidyl (meth)acrylate composition of the present invention within the above-described range, it is possible to appropriately suppress a reaction between the quaternary ammonium salt and a phenolic polymerization inhibitor, thereby ensuring the long-term storage stability of the glycidyl (meth)acrylate composition. In a preferred embodiment of the present invention, the content of the quaternary ammonium salt in the glycidyl (meth)acrylate composition may be 30 ppm or less.

The quaternary ammonium salt is as described above. Specifically, in the method for suppressing deactivation of a phenolic polymerization inhibitor in a glycidyl (meth)acrylate composition of the present invention, examples of the quaternary ammonium salt include: tetraalkyl ammonium halogenides such as tetramethylammonium chloride, trimethylethylammonium chloride, dimethyl diethyl ammonium chloride, triethylmethylammonium chloride, and tetraethylammonium chloride; and trialkylbenzylammonium halogenides such as trimethylbenzylammonium chloride and triethylbenzylammonium chloride. The quaternary ammonium salt may be one kind or two or more kinds thereof. However, among the above, tetramethylammonium chloride, triethylmethylammonium chloride, tetraethylammonium chloride, triethylbenzylammonium chloride, and trimethylbenzylammonium chloride are preferable. In a preferred embodiment of the method of the present invention, the quaternary ammonium salt which may be present in the glycidyl (meth)acrylate composition is tetraalkylammonium halogenide. In a more preferred embodiment of the method of the present invention, the quaternary ammonium salt which may be present in the glycidyl (meth)acrylate composition is tetramethylammonium chloride or triethylmethylammonium chloride.

The strong acid salt is as described above. Specifically, the strong acid salt is not particularly limited as long as it can suppress deactivation of the phenolic polymerization inhibitor present in the glycidyl (meth)acrylate composition. Examples thereof include a sulfonate, a nitrate, and a phosphate. In the method for suppressing deactivation of a phenolic polymerization inhibitor in a glycidyl (meth)acrylate resin composition of the present invention, the strong acid salt is selected from the group consisting of sodium salts, calcium salts, potassium salts, and magnesium salts of the above-described strong acid. In one embodiment of the present invention, the strong acid salt may be a sodium salt of the above-described strong acid. In another embodiment of the present invention, the strong acid salt may be a calcium salt of the above-described strong acid. In a preferred embodiment of the present invention, the strong acid salt is a sodium salt of the above-described strong acid.

In one embodiment of the method for suppressing deactivation of a phenolic polymerization inhibitor in a glycidyl (meth)acrylate resin composition of the present invention, the strong acid salt may be a sulfonate. In a preferred embodiment of the present invention, the strong acid salt may be alkyl benzene sulfonate or alkyl sulfonate. In a preferred embodiment of the present invention, the strong acid salt may be, for example, sodium alkylbenzene sulfonate, potassium alkylbenzene sulfonate, calcium bis(alkylbenzenesulfonate), magnesium bis(alkylbenzenesulfonate), sodium alkylsulfonate, potassium alkylsulfonate, calcium bis(alkylsulfonate), or magnesium bis(alkylsulfonate). In a more preferred embodiment of the present invention, the strong acid salt may be, for example, p-toluenesulfonate, methanesulfonate, laurylsulfonate, dodecylbenzenesulfonate, or benzenesulfonate. In a further preferred embodiment of the present invention, the strong acid salt is sodium p-toluenesulfonate or sodium methanesulfonate.

In another embodiment of the method for suppressing deactivation of a phenolic polymerization inhibitor in a glycidyl (meth)acrylate resin composition of the present invention, the strong acid salt may be a nitrate. In a preferred embodiment of the present invention, the strong acid salt may be, for example, sodium nitrate, calcium nitrate, potassium nitrate, or magnesium nitrate. In a more preferred embodiment of the present invention, the strong acid may be sodium nitrate.

In another embodiment of the method for suppressing deactivation of a phenolic polymerization inhibitor in a glycidyl (meth)acrylate resin composition of the present invention, the strong acid salt may be a phosphate In a more preferred embodiment of the present invention, the strong acid salt may be, for example, sodium phosphate, calcium phosphate, potassium phosphate, or magnesium phosphate. In a further preferred embodiment of the present invention, the strong acid salt is sodium phosphate.

In one embodiment of the method for suppressing deactivation of a phenolic polymerization inhibitor in a glycidyl (meth)acrylate resin composition of the present invention, the content of the strong acid salt in the glycidyl (meth)acrylate composition is adjusted to 0.50 equivalents or more relative to the amount of the quaternary ammonium salt by mole. The content of the strong acid salt in the glycidyl (meth)acrylate composition may be adjusted to, for example, 0.50 equivalents, 0.75 equivalents, 1.00 equivalent, 1.25 equivalents, 1.50 equivalents, 1.75 equivalents, 2.00 equivalents, 2.50 equivalents, or 3.00 equivalents relative to the amount of the quaternary ammonium salt by mole. The content of the strong acid salt in the glycidyl (meth)acrylate composition is adjusted to preferably 0.50 equivalents or more, more preferably 0.75 equivalents or more, still more preferably 1.00 equivalent or more relative to the amount of the quaternary ammonium salt by mole. In one preferred embodiment of the present invention, the method for suppressing deactivation of a phenolic polymerization inhibitor in a glycidyl (meth)acrylate resin composition comprises adjusting the content of the strong acid salt in the glycidyl (meth)acrylate composition to 0.50 equivalents or more relative to the amount of the quaternary ammonium salt by mole. In one more preferred embodiment of the present invention, the method for suppressing deactivation of a phenolic polymerization inhibitor in a glycidyl (meth)acrylate resin composition comprises adjusting the content of the strong acid salt in the glycidyl (meth)acrylate composition to 0.75 equivalents or more relative to the amount of the quaternary ammonium salt by mole. In one further preferred embodiment of the present invention, the method for suppressing deactivation of a phenolic polymerization inhibitor in a glycidyl (meth)acrylate resin composition comprises adjusting the content of the strong acid salt in the glycidyl (meth)acrylate composition to 1.00 equivalent or more relative to the amount of the quaternary ammonium salt by mole. In an embodiment of the present invention, the content of the strong acid salt in the glycidyl (meth)acrylate composition can be appropriately adjusted to, for example, 1.50 equivalents or less, 1.75 equivalents or less, 2.00 equivalents or less, 2.50 equivalents or less, 3.00 equivalents or less, or 5.00 equivalents or less relative to the amount of the quaternary ammonium salt by mole.

The phenolic polymerization inhibitor is as described above. Specifically, in the method for suppressing deactivation of a phenolic polymerization inhibitor in a glycidyl (meth)acrylate composition of the present invention, examples of the phenolic polymerization inhibitor include, but are not limited to, p-methoxyphenol (hereinafter also referred to as “MQ”), hydroquinone, 2,6-di-tert-butyl-4-methylphenol, 2,2′-methylene-bis(4-methyl-6-tert-butylphenol), and Topanol A (2-(tert-butyl)-4,6-dimethylphenol). In an embodiment of the present invention, the phenolic polymerization inhibitor is preferably p-methoxyphenol, hydroquinone, or Topanol A (2-(tert-butyl)-4,6-dimethylphenol), more preferably p-methoxyphenol or hydroquinone, most preferably p-methoxyphenol.

The amount of the phenolic polymerization inhibitor used in producing glycidyl (meth)acrylate to be added is generally in a range of 0.0005 to 0.01 equivalents with respect to the amount of (meth)acryloyl group by mole. The content of the phenolic polymerization inhibitor present in the produced glycidyl (meth)acrylate composition is in a range of 20 to 200 ppm, preferably 20 to 150 ppm.

In the method for suppressing deactivation of a phenolic polymerization inhibitor in a glycidyl (meth)acrylate composition of the present invention, by adjusting the content of the quaternary ammonium salt present in the glycidyl (meth)acrylate composition within a certain range as described above, it is possible to appropriately suppress the reaction between the quaternary ammonium salt and the phenolic polymerization inhibitor.

A glycidyl (meth)acrylate composition is generally produced by performing purification by distillation of a reaction mixture obtained by the reaction of epichlorohydrin with (meth)acrylic acid or a metal salt of (meth)acrylic acid. The contents of the quaternary ammonium salt and the strong acid salt in the glycidyl (meth)acrylate composition are adjusted based on the amount of the quaternary ammonium salt used during production and the distillation method and conditions for distilling and recovering glycidyl (meth)acrylate.

The amount of the quaternary ammonium salt added during production is preferably 0.0001 to 0.01 equivalents with respect to the amount of (meth)acryloyl group by mole. The amount of the strong acid salt added during production is preferably 0.5 to 3.0 equivalents relative to the amount of the quaternary ammonium salt by mole.

Examples of the distillation method include simple distillation and rectification, and the reflux ratio in rectification is preferably 0.1 to 3.0. The distillation conditions include temperature and pressure, and the temperature is preferably 40° C. to 120° C., and the pressure is preferably 0.05 to 10 kPaA.

For example, the “number of days required for a phenolic polymerization inhibitor to deteriorate by 10%” and the “reaction rate constant” can be used as indexes for suppressing deactivation of the phenolic polymerization inhibitor.

The “number of days required for a phenolic polymerization inhibitor to deteriorate by 10%” (unit: day) refers to the number of days required for a phenolic polymerization inhibitor present in the produced glycidyl (meth)acrylate composition to be deactivated by 10%. In the method of the present invention, the “number of days required for a phenolic polymerization inhibitor to deteriorate by 10%” is preferably 20 days or more, more preferably 50 days or more, still more preferably 60 days or more, most preferably 90 days or more. As long as the “number of days required for a phenolic polymerization inhibitor to deteriorate by 10%” is within the above-described range, it can be said that deactivation of the phenolic polymerization inhibitor in the glycidyl (meth)acrylate composition is appropriately being suppressed. In addition, the “number of days required for a phenolic polymerization inhibitor to deteriorate by 10%” is preferably twice or more, more preferably three times or more, still more preferably five times or more, most preferably ten times or more, compared to the case of not adding a strong acid salt.

The “reaction rate constant” (unit: day⁻¹) is a constant for the rate of deterioration of a phenolic polymerization inhibitor, which corresponds to k in the following Formula (1).

−d[I]/dt=k[I] (1)

Here, [I] refers to a phenolic polymerization inhibitor concentration. The deterioration of the phenolic polymerization inhibitor is due to the reaction with glycidyl (meth)acrylate. Thus, initially, the concentration of glycidyl (meth)acrylate should be considered for calculating the reaction rate; however, the concentration of glycidyl (meth)acrylate is regarded as constant because glycidyl (meth)acrylate contained in the glycidyl (meth)acrylate composition is in excess of the phenolic polymerization inhibitor. In the method of the present invention, the “reaction rate constant” is preferably 5.3×10⁻³ day⁻¹ or less, more preferably 2.1×10⁻³ day⁻¹ or less, still more preferably 1.8×10⁻³ day⁻¹ or less, most preferably 1.2×10⁻³ day⁻¹ or less. As long as the “reaction rate constant” is within the above-described range, it can be said that deactivation of the phenolic polymerization inhibitor in the glycidyl (meth)acrylate composition is appropriately being suppressed.

### EXAMPLES

Hereinafter, the present invention will be specifically described with reference to the following examples. However, these examples are not intended to limit the present invention.

### Reference Example 1

Glycidyl methacrylate with a purity of 99.5% (hereinafter sometimes referred to as “GMA”) in an amount of 40.0 g was mixed with 10.0 g of pure water and stirred for 30 seconds with a vortex mixer, thereby dissolving the salt component in GMA in the aqueous phase. An aqueous phase was recovered from the mixture, and ion components in the aqueous phase were confirmed.

Specifically, measurements were carried out under the following conditions using cation ion chromatography and anion ion chromatography.

### <Cation Ion Chromatography>

Column: Shodex IC YS-50 (inner diameter: 4.6 mm; length 125 mm)

Column temperature: 40° C.

Eluent: 0.2 mmol/L nitric acid aqueous solution

Flow rate: 0.8 mL/min

Detector: Electric conductivity detector

Sample injection volume: 100 μL

### <Anion Ion Chromatography>

Column: Tosoh TSKgel IC-Anion-PW (inner diameter: 4.6 mm; length: 50 mm)

Column temperature: 40° C.

Eluent: Tosoh TSKgel eluent IC-Anion-A

Flow rate: 0.8 mL/min

Detector: Electric conductivity detector

Sample injection volume: 100 μL

Analysis by cation ion chromatography and anion ion chromatography showed no peaks detected, thereby confirming that the produced GMA did not contain a salt component such as a quaternary ammonium salt.

### Reference Example 2

A predetermined amount of p-methoxyphenol (special grade reagent of FUJIFILM Wako Pure Chemical Corporation) was added to GMA of Reference Example 1 to prepare a test solution. The test solution was stored at 25° C. under ordinary pressure in the atmosphere to confirm the MQ concentration decrease. The concentration of p-methoxyphenol (MQ) in GMA was quantitatively determined using high-performance liquid chromatography.

<Quantitative Determination of p-Methoxyphenol (High-Performance Liquid Chromatography)>

Column: Tosoh TSKgel ODS-120T (particle diameter: 5 μm; inner diameter: 4.6 mm; length: 25 cm)

Column temperature: 40° C.

Eluent: Acetonitrile/pure water/acetic acid=700/300/1 (volume ratio)

Flow rate: 0.8 mL/min

Detector: UV-visible spectrometer (wavelength: 285 nm)

Sample injection volume: 5 μL

Retention time: MQ (4.5 min)

The MQ concentration at the start of testing was 102.4 ppm, while the MQ concentration after storage for 90 days was 102.1 ppm, showing substantially no deterioration (deactivation) of MQ.

### Comparative Example 1

A predetermined amount of MQ and 5.00 ppm of triethylmethylammonium chloride (“EMAC”) were added to GMA of Reference Example 1 to prepare a test solution. The MQ concentration of the test solution was 101.8 ppm. The test solution was stored at 25° C. under ordinary pressure in the atmosphere. The MQ concentration was quantitatively determined in the same manner as Reference Example 2. Accordingly, the MQ concentrations after storage for 15 days, 34 days, 49 days, and 61 days were 92.4 ppm, 77.0 ppm, 65.8 ppm, and 58.2 ppm, respectively. The reaction rate constant calculated in the same manner as Example 3 was 9.32×10⁻³ day⁻¹, and the time required for MQ to deteriorate by 10% was 11 days.

### Example 1

To the test solution prepared in Comparative Example 1, sodium p-toluenesulfonate (special grade reagent of FUJIFILM Wako Pure Chemical Corporation, “p-TSANa”) was added in an amount of 0.50 equivalents relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 97.6 ppm, 91.4 ppm, 86.5 ppm, and 82.7 ppm, respectively.

When ln([MQ]/[MQ]ₙ) was plotted against time for the obtained results, a linear relationship was obtained. From the above, the deterioration of MQ was a primary reaction, and the reaction rate constant was 3.43×10⁻³ day⁻¹. From the calculated reaction rate constant, the time required for MQ to deteriorate by 10% was calculated, resulting in 31 days. The addition of sodium p-toluenesulfonate caused the rate of deterioration of MQ to decrease. [MQ]0 is the molar concentration of MQ at the start of testing, and [MQ] is the molar concentration of MQ at the time of measurement.

### Example 2

To the test solution prepared in Comparative Example 1, sodium p-toluenesulfonate (special grade reagent of FUJIFILM Wako Pure Chemical Corporation, “p-TSANa”) was added in an amount of 0.75 equivalents relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 99.7 ppm, 97.2 ppm, 95.9 ppm, and 94.8 ppm, respectively. The reaction rate constant calculated in the same manner as Example 3 was 1.18×10⁻³ day⁻¹, and the time required for MQ to deteriorate by 10% was 89 days. The addition of sodium p-toluenesulfonate caused the rate of deterioration of MQ to decrease.

### Example 3

To the test solution prepared in Comparative Example 1, sodium p-toluenesulfonate (special grade reagent of FUJIFILM Wako Pure Chemical Corporation, “p-TSANa”) was added in an amount of 1.00 equivalent relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 100.4 ppm, 99.1 ppm, 99.0 ppm, and 99.2 ppm, respectively. The reaction rate constant calculated in the same manner as Example 3 was 4.36×10⁻⁴ day⁻¹, and the time required for MQ to deteriorate by 10% was 242 days. The addition of sodium p-toluenesulfonate caused the rate of deterioration of MQ to decrease.

### Example 4

To the test solution prepared in Comparative Example 1, sodium p-toluenesulfonate (special grade reagent of FUJIFILM Wako Pure Chemical Corporation, “p-TSANa”) was added in an amount of 1.25 equivalents relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 101.3 ppm, 100.3 ppm, 100.3 ppm, and 100.6 ppm, respectively. The reaction rate constant calculated in the same manner as Example 3 was 2.39×10⁻⁴ day⁻¹, and the time required for MQ to deteriorate by 10% was 442 days. The addition of sodium p-toluenesulfonate caused the rate of deterioration of MQ to decrease.

### Example 5

To the test solution prepared in Comparative Example 1, sodium p-toluenesulfonate (special grade reagent of FUJIFILM Wako Pure Chemical Corporation, “p-TSANa”) was added in an amount of 1.50 equivalents relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 101.3 ppm, 100.4 ppm, 100.4 ppm, and 100.8 ppm, respectively. The reaction rate constant calculated in the same manner as Example 3 was 2.05×10⁻⁴ day⁻¹, and the time required for MQ to deteriorate by 10% was 515 days. The addition of sodium p-toluenesulfonate caused the rate of deterioration of MQ to decrease.

### Example 6

To the test solution prepared in Comparative Example 1, sodium methanesulfonate (special grade reagent of FUJIFILM Wako Pure Chemical Corporation, “Me-SO₃Na”) was added in an amount of 1.00 equivalent relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 96.1 ppm, 88.5 ppm, 83.9 ppm, and 81.1 ppm, respectively. The reaction rate constant calculated in the same manner as Example 3 was 3.81×10⁻³ day⁻¹, and the time required for MQ to deteriorate by 10% was 28 days. The addition of sodium methanesulfonate caused the rate of deterioration of MQ to decrease.

### Example 7

To the test solution prepared in Comparative Example 1, sodium nitrate (special grade reagent of FUJIFILM Wako Pure Chemical Corporation, NaNO₃) was added in an amount of 1.00 equivalent relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 94.2 ppm, 84.0 ppm, 78.2 ppm, and 74.9 ppm, respectively. The reaction rate constant calculated in the same manner as Example 3 was 5.16×10⁻³ day⁻¹, and the time required for MQ to deteriorate by 10% was 20 days. The addition of sodium nitrate caused the rate of deterioration of MQ to decrease.

### Comparative Example 2

To the test solution prepared in Comparative Example 1, sodium acetate (special grade reagent of FUJIFILM Wako Pure Chemical Corporation, AcONa) was added in an amount of 1.00 equivalent relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 101.8 ppm, while the MQ concentrations after storage for 15 days, 34 days, 49 days, and 61 days were 92.9 ppm, 78.4 ppm, 67.9 ppm, and 60.6 ppm, respectively. The reaction rate constant calculated in the same manner as Example 3 was 8.63×10⁻³ day⁻¹, and the time required for MQ to deteriorate by 10% was 12 days. Even the addition of sodium nitrate did not substantially cause the rate of deterioration of MQ to change.

### Example 8

To GMA of Reference Example 1, a predetermined amount of MQ and 1.00 ppm of triethylmethylammonium chloride (“EMAC”) were added. Then, sodium p-toluenesulfonate (special grade reagent of FUJIFILM Wako Pure Chemical Corporation, “p-TSANa”) was added in an amount of 0.50 equivalents relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 99.3 ppm, while the MQ concentrations after storage for 10 days, 21 days, 32 days, 46 days, and 65 days were 98.2 ppm, 97.5 ppm, 96.7 ppm, 95.3 ppm, and 94.2 ppm, respectively. The reaction rate constant calculated in the same manner as Example 3 was 8.15×10⁻⁴ day⁻¹, and the time required for MQ to deteriorate by 10% was 129 days. The addition of sodium p-toluenesulfonate caused the rate of deterioration of MQ to decrease.

### Example 9

To GMA of Reference Example 1, a predetermined amount of MQ and 0.75 ppm of triethylmethylammonium chloride (“EMAC”) were added. Then, sodium p-toluenesulfonate (special grade reagent of FUJIFILM Wako Pure Chemical Corporation, “p-TSANa”) was added in an amount of 1.00 equivalent relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 99.3 ppm, while the MQ concentrations after storage for 10 days, 21 days, 32 days, 46 days, and 65 days were 98.8 ppm, 98.5 ppm, 98.1 ppm, 98.0 ppm, and 97.2 ppm, respectively. The reaction rate constant calculated in the same manner as Example 3 was 3.08×10⁻⁴ day⁻¹, and the time required for MQ to deteriorate by 10% was 342 days. The addition of sodium p-toluenesulfonate caused the rate of deterioration of MQ to decrease.

### Example 10

To GMA of Reference Example 1, a predetermined amount of MQ and 1.00 ppm of triethylmethylammonium chloride (“EMAC”) were added. Then, sodium p-toluenesulfonate (special grade reagent of FUJIFILM Wako Pure Chemical Corporation, “p-TSANa”) was added in an amount of 1.00 equivalent relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 99.3 ppm, while the MQ concentrations after storage for 10 days, 21 days, 32 days, 46 days, and 65 days were 98.2 ppm, 97.5 ppm, 96.7 ppm, 95.3 ppm, and 94.2 ppm, respectively. The reaction rate constant calculated in the same manner as Example 3 was 1.35×10⁻⁴ day⁻¹, and the time required for MQ to deteriorate by 10% was 781 days. The addition of sodium p-toluenesulfonate caused the rate of deterioration of MQ to decrease.

### Example 11

To the test solution prepared in Comparative Example 3, sodium p-toluenesulfonate (special grade reagent of FUJIFILM Wako Pure Chemical Corporation, “p-TSANa”) was added in an amount of 1.00 equivalent relative to the amount of tetramethylammonium chloride (“TMAC”) by mole and stored at 25° C. under ordinary pressure in the atmosphere. The MQ concentration was quantitatively determined in the same manner as Reference Example 2. Accordingly, the MQ concentration at the start of testing was 99.6 ppm, while the MQ concentrations after storage for 10 days, 21 days, 32 days, 46 days, and 65 days were 99.4 ppm, 99.3 ppm, 99.1 ppm, 99.1 ppm, and 98.8 ppm, respectively. The reaction rate constant calculated in the same manner as Example 3 was 1.11×10⁻⁴ day⁻¹, and the time required for MQ to deteriorate by 10% was 948 days. The addition of sodium p-toluenesulfonate caused the rate of deterioration of MQ to decrease.

### Example 12

To the test solution prepared in Comparative Example 4, sodium p-toluenesulfonate (special grade reagent of FUJIFILM Wako Pure Chemical Corporation, “p-TSANa”) was added in an amount of 1.00 equivalents relative to the amount of triethylmethylammonium chloride (“EMAC”) by mole and stored at 25° C. under ordinary pressure in the atmosphere. The MQ concentration was quantitatively determined in the same manner as Reference Example 2. Accordingly, the MQ concentration at the start of testing was 50.1 ppm, while the MQ concentrations after storage for 10 days, 21 days, 32 days, 46 days, and 65 days were 49.9 ppm, 49.9 ppm, 49.6 ppm, 49.4 ppm, and 49.1 ppm, respectively. The reaction rate constant calculated in the same manner as Example 3 was 3.04×10⁻⁴ day⁻¹, and the time required for MQ to deteriorate by 10% was 347 days. The addition of sodium p-toluenesulfonate caused the rate of deterioration of MQ to decrease.

The results obtained in the Reference Examples, Examples, and Comparative Examples are shown in Tables 1 and 2 below.

TABLE 1

| | Reference | Comparative | | | | |
|---|---|---|---|---|---|---|
| | Example 2 | Example 1 | Example 1 | Example 2 | Example 3 | Example 4 |
| Quaternary ammonium salt | — | EMAC | EMAC | EMAC | EMAC | EMAC |
| (ppm) | 0 | 5.00 | 5.00 | 5.00 | 5.00 | 5.00 |
| Strong acid salt | — | — | p-TSANa | p-TSANa | p-TSANa | p-TSANa |
| Strong acid salt/Quaternary | 0 | 0 | 0.50 | 0.75 | 1.00 | 1.25 |
| ammonium salt | | | | | | |
| (mol/mol) | | | | | | |
| Initial concentration of phenolic | MQ | MQ | MQ | MQ | MQ | MQ |
| polymerization inhibitor | | | | | | |
| (ppm) | 102.4 | 101.8 | 101.8 | 101.8 | 101.8 | 101.8 |
| Reaction rate constant (day⁻¹) | 5.97E−05 | 9.32E−03 | 3.43E−03 | 1.18E−03 | 4.36E−04 | 2.39E−04 |
| Number of days required for | 1764 | 11 | 31 | 89 | 242 | 442 |
| phenolic polymerization inhibitor | | | | | | |
| to deteriorate (day) | | | | | | |

| | | | | Comparative |
|---|---|---|---|---|
| | Example 5 | Example 6 | Example 7 | Example 2 |
| Quaternary ammonium salt | EMAC | EMAC | EMAC | EMAC |
| (ppm) | 5.00 | 5.00 | 5.00 | 5.00 |
| Strong acid salt | p-TSANa | Me—SO₃Na | NaNO₃ | AcONa |
| Strong acid salt/Quaternary | 1.50 | 1.00 | 1.00 | 1.00 |
| ammonium salt | | | | |
| (mol/mol) | | | | |
| Initial concentration of phenolic | MQ | MQ | MQ | MQ |
| polymerization inhibitor | | | | |
| (ppm) | 101.8 | 101.8 | 101.8 | 101.8 |
| Reaction rate constant (day⁻¹) | 2.05E−04 | 3.81E−03 | 5.16E−03 | 8.63E−03 |
| Number of days required for | 515 | 28 | 20 | 12 |
| phenolic polymerization inhibitor | | | | |
| to deteriorate (day) | | | | |

TABLE 2

| | Example 8 | Example 9 | Example 10 | Example 11 | Example 12 |
|---|---|---|---|---|---|
| Quaternary ammonium salt | EMAC | EMAC | EMAC | TMAC | EMAC |
| (ppm) | 1.00 | 1.00 | 1.00 | 1.00 | 1.00 |
| Strong acid salt | p-TSANa | p-TSANa | p-TSANa | p-TSANa | p-TSANa |
| Strong acid salt/Quaternary | 0.50 | 0.75 | 1.00 | 1.00 | 1.00 |
| ammonium salt | | | | | |
| (mol/mol) | | | | | |
| Initial concentration of phenolic | MQ | MQ | MQ | MQ | MQ |
| polymerization inhibitor | | | | | |
| (ppm) | 99.3 | 99.3 | 99.3 | 99.6 | 50.1 |
| Reaction rate constant (day⁻¹) | 8.15E−04 | 3.08E−04 | 1.35E−04 | 1.11E−04 | 3.04E−04 |
| Number of days required for | 129 | 342 | 781 | 948 | 347 |
| phenolic polymerization inhibitor | | | | | |
| to deteriorate (day) | | | | | |

Abbreviations in the table are as follows:

EMAC: Triethylmethylammonium chloride

TMAC: Tetramethylammonium chloride

MQ: p-Methoxyphenol

p-TSANa: Sodium p-toluenesulfonate

Me-SO₃Na: Sodium methanesulfonate

NaNO₃: Sodium nitrate

AcONa: Sodium acetate

As described above, each example of the glycidyl (meth)acrylate composition of the present invention is a glycidyl (meth)acrylate composition, which includes a phenolic polymerization inhibitor that is unlikely to deteriorate such that the glycidyl (meth)acrylate composition can be stably stored for a long period of time. In addition, it is possible to appropriately suppress the deterioration (deactivation) of a phenolic polymerization inhibitor contained in a glycidyl (meth)acrylate composition using the method of the present invention. The glycidyl (meth)acrylate composition and the method of the present invention can contribute to ensuring the long-term storage stability of a glycidyl (meth)acrylate composition.

1. A method for suppressing deactivation of a phenolic polymerization inhibitor in a glycidyl (meth)acrylate composition, comprising adjusting a content of a strong acid salt in the glycidyl (meth)acrylate composition to 0.50 equivalents or more relative to an amount of a quaternary ammonium salt by mole.

2. The method according to claim 1, wherein the strong acid salt is selected from the group consisting of a sulfonate, a nitrate, and a phosphate.

3. The method according to claim 2, wherein the strong acid salt is alkyl benzene sulfonate or alkyl sulfonate.

4. The method according to claim 3, wherein the strong acid salt is sodium p-toluenesulfonate or sodium methanesulfonate.

5. The method according to claim 2, wherein the strong acid salt is sodium nitrate.

6. The method according to claim 1, wherein the quaternary ammonium salt is tetraalkylammonium halogenide.

7. The method according to claim 6, wherein the quaternary ammonium salt is tetramethylammonium chloride or triethylmethylammonium chloride.

8. The method according to claim 1, wherein the phenolic polymerization inhibitor is p-methoxyphenol, hydroquinone, or Topanol A (2-(tert-butyl)-4,6-di methyl phenol).

9. The method according to claim 1, wherein the glycidyl (meth)acrylate composition comprises the strong acid salt in an amount of 0.50 equivalents or more relative to the amount of the quaternary ammonium salt by mole.

10. The method according to claim 1, wherein the glycidyl (meth)acrylate is glycidyl methacrylate.

11. A glycidyl (meth)acrylate composition comprising a glycidyl (meth)acrylate, a quaternary ammonium salt, a strong acid salt, and a phenolic polymerization inhibitor.

12. The glycidyl (meth)acrylate composition according to claim 11, wherein the strong acid salt is selected from the group consisting of a sulfonate, a nitrate, and a phosphate.

13. The glycidyl (meth)acrylate composition according to claim 12, wherein the strong acid salt is alkyl benzene sulfonate or alkyl sulfonate.

14. The glycidyl (meth)acrylate composition according to claim 13, wherein the strong acid salt is sodium p-toluenesulfonate or sodium methanesulfonate.

15. The glycidyl (meth)acrylate composition according to claim 12, wherein the strong acid salt is sodium nitrate.

16. The glycidyl (meth)acrylate composition according to claim 11, wherein the quaternary ammonium salt is tetraalkylammonium halogenide.

17. The glycidyl (meth)acrylate composition according to claim 16, wherein the quaternary ammonium salt is tetramethylammonium chloride or triethylmethylammonium chloride.

18. The glycidyl (meth)acrylate composition according to claim 11, wherein the phenolic polymerization inhibitor is p-methoxyphenol, hydroquinone, or Topanol A (2-(tert-butyl)-4,6-dimethylphenol).

19. The glycidyl (meth)acrylate composition according to claim 11, which comprises the strong acid salt in an amount of 0.50 equivalents or more relative to the amount of the quaternary ammonium salt by mole.

20. The glycidyl (meth)acrylate composition according to claim 11, wherein the glycidyl (meth)acrylate is glycidyl methacrylate.
